(19) United States
(12) Patent Application Publication
(10) Pub. No.: US 2005/0210615 A1
(43) Pub. Date: Sep. 29, 2005

(54) Oral care method

(76) Inventors: Ramachandra Shastry, Dayton, NJ (US); Yelloji Rao Mirajkar, Piscataway, NJ (US); Nebojsa Milanovich, Somerset, NJ (US); Nagaraj Dixit, Plainsboro, NJ (US); Michael Prencipe, West Windsor, NJ (US)

Correspondence Address: Harness, Dickey & Pierce, P.L.C., Suite 400, 7700 Bonhomme, Clayton, MO, 63105, US

(21) Appl. No.: 10/811,723

(22) Filed: Mar. 29, 2004

(51) Int. Cl. A61K 7/16; A47L 23/04

(52) U.S. Cl. 15/210.1; 424/49

(57) ABSTRACT

A method for improving effectiveness of an oral care agent comprises preconditioning an oral surface by wiping the surface with an absorbent fabric having impregnated therein or coated thereon an orally acceptable preconditioning agent such as an activating agent for the oral care agent, wherein the wiping transfers an activating effective amount of the preconditioning agent from the fabric to the oral surface, and thereafter applying a composition comprising the oral care agent to the oral surface.

### FIELD

This invention relates to a method for oral care, more particularly to a method for care of a dental surface.

### BACKGROUND

Certain agents useful in oral care, for example in cleaning, whitening and protecting teeth, exhibit enhanced performance when acting in presence of an activating agent. As an example, effectiveness of a peroxy compound in whitening a dental surface can be enhanced in presence of certain activating agents, such as manganese coordination complex compounds and iron and copper salts. Another class of activating agent for peroxy compounds is a basifying agent, typically an alkaline compound such as a carbonate or bicarbonate salt, it being well known that release of oxygen from peroxy compounds such as hydrogen peroxide is promoted at higher pH. As a further example, a bicarbonate salt used to provide a clean mouth feel and/or other benefits through effervescence in the mouth can be enhanced in presence of an acidifying agent, which accelerates such effervescence. As a still further example, incorporation of fluoride ions into tooth enamel from a soluble fluoride source such as sodium fluoride can be enhanced in presence of a calcium salt and/or an acidifying agent.

In these and other situations, premature interaction of the oral care agent with the activating agent can cause partial or total loss of effectiveness. For example, if a peroxy compound is formulated for storage in an alkaline medium, its effectiveness as a whitening agent can be reduced through premature release of oxygen. Similarly, exposure of sodium bicarbonate to an acid medium prior to use results in premature release of carbon dioxide and loss of efficacy as an effervescent agent when placed in the mouth. Coformulation of sodium fluoride with a calcium salt leads to formation of insoluble calcium fluoride and consequent reduction in availability of fluoride ion for uptake when applied following a period of storage to teeth.

Efforts to deliver an oral care agent and activating agent therefor to the mouth have included simultaneous delivery of such agents in two-component products wherein the agents are kept separate until introduction to the mouth or shortly prior thereto.

For example, U.S. Pat. No. 5,648,064 to Gaffar & Fakhry-Smith discloses a two-component whitening dentifrice composition wherein one component comprises a peroxygen compound and the other a manganese coordination complex such as manganese gluconate. The two components are maintained separate from each other until dispensed for use.

U.S. Pat. No. 6,254,857 to Hoic et al. discloses a two-component whitening dentifrice composition wherein one component comprises a peroxide compound and the other a mixture of iron and copper salts. The two components are maintained separate from each other until dispensed for use.

U.S. Patent Application Publication No. 2002/0141949 of Banerjee & Friedman proposes applying a gel comprising a peroxide bleaching agent to a tooth surface using an applicator such as a brush having an activator (e.g., manganous chloride, manganous citrate, ferrous sulfate, sodium carbonate, sodium bicarbonate or catalase) in a dry form stored therein.

European Patent No. 0 897 714 discloses a two-component mouthwash product wherein one component comprises hydrogen peroxide in aqueous solution at pH <4.5 and the other a buffer salt in aqueous or aqueous/alcoholic solution at pH >8. The two components are mixed before use.

U.S. Pat. No. 5,928,628 to Pellico discloses a two-component bleaching system adapted for application to teeth from a dental bleaching tray. One component comprises a peroxide gel having a pH of about 4 to about 7 and the other an alkaline gel having a pH of about 9 to about 13. The two components are mixed before use.

U.S. Pat. No. 4,487,757 to Kiozpeoplou proposes segregating, in separate sections of a toothpaste container, two portions of toothpaste, one comprising a stabilized alkali metal bicarbonate and the other an acid or acid salt reactive with the bicarbonate. The toothpaste is said to effervesce strongly during intimate mixing of the two portions when toothbrushing.

U.S. Pat. No. 5,045,305 to Clarkson et al. describes an oral care product comprising two separate toothpaste or mouthwash components, one comprising sodium fluoride and the other calcium chloride. The components are mixed before being introduced to the mouth.

U.S. Pat. No. 6,346,235 to Joziak et al. discloses a method for enhancing fluoride availability using a two-component dentifrice wherein one component comprises sodium fluoride in an alkaline environment and the other a source of phosphate ions in an acid environment.

In contrast to the above disclosures, U.S. Pat. No. 6,174,516 to Curtis et al. discloses a sequential treatment method. According to the '516 patent there is first applied to teeth an aqueous rinse composition having an alkaline pH and thereafter the teeth are brushed with a peroxide dentifrice.

U.S. Pat. No. 5,133,971 to Copelan & Copelan discloses a membrane, for example an unwoven cellulose fiber mat, having impregnated therein a dehydrated composition comprising combinations of agents useful in dental hygiene, such as sodium bicarbonate.

Patents and publications cited above are incorporated herein by reference.

There remains in the art a need for alternative methods for activating an oral care agent in the mouth by interaction of the oral care agent with an activating agent.

### SUMMARY

Now provided is a method for improving effectiveness of an oral care agent, the method comprising preconditioning an oral surface by wiping the surface with an absorbent fabric having impregnated therein or coated thereon an orally acceptable preconditioning agent, wherein the wiping transfers an effective amount of the preconditioning agent from the fabric to the oral surface, and thereafter applying a composition comprising the oral care agent to the oral surface.

According to an embodiment of the invention the preconditioning agent is an activating agent for the oral care agent, and the wiping transfers an activating effective amount of the activating agent from the fabric to the oral surface.

In a particular embodiment there is provided a method for whitening a dental surface, the method comprising wiping the surface with an absorbent fabric having impregnated therein or coated thereon an orally acceptable basifying agent, and thereafter applying a composition comprising an orally acceptable peroxy compound to the surface.

There is further provided a kit useful in practice of the method of the invention. The kit comprises (a) a composition comprising an oral care agent and (b) an absorbent fabric having impregnated therein or coated thereon an orally acceptable preconditioning agent, for example an activating agent for the oral care agent. In a particular embodiment such a kit comprises (a) a composition comprising an orally acceptable peroxy compound and (b) an absorbent fabric having impregnated therein or coated thereon an orally acceptable basifying agent.

A further embodiment of the invention is an article useful in preconditioning a dental surface for whitening treatment, the article comprising a moist towelette carrying in aqueous solution an orally acceptable basifying agent in an amount effective to provide a pH of at least about 7 at the dental surface upon wiping the surface with the towelette.

### DETAILED DESCRIPTION

An “oral care” agent herein is an agent useful in topical treatment or prophylaxis of a disease, disorder or unwanted condition of the mouth, in promotion of oral hygiene or in cosmetic enhancement of an oral surface. Such agents can be applied generally to the interior of the mouth, or to particular portions thereof, such as the tongue, palate, buccal mucosa, teeth and/or gums.

An “oral surface” herein encompasses any soft or hard surface within the mouth including surfaces of the tongue, hard and soft palate, buccal mucosa, gums and dental surfaces.

A “dental surface” herein is a surface of a natural tooth or a hard surface of artificial dentition including a crown, cap, filling, bridge, denture, dental implant and the like.

“Effectiveness” of an oral care agent herein includes one or more of (a) degree of effectiveness achieved, (b) speed with which a given degree of effectiveness is achieved and (c) duration of effect (a property sometimes referred to as “substantivity”). Effectiveness can be measured in absolute terms or in relative terms, for example by comparison with an untreated control or a standard treatment.

The term “preconditioning” herein with respect to an oral or dental surface means affecting the surface or its immediate environment in such a way as to make the surface more receptive and/or responsive to a subsequently applied oral care agent.

“Wiping” a surface with a fabric can, but does not necessarily, involve lateral movement of the fabric against the surface, i.e., a rubbing action. The term “wiping” herein also embraces simple swabbing, i.e., single or repeated contact of the surface with the fabric without significant rubbing.

An “activating agent” herein is an agent that promotes effectiveness of an oral care agent. For example, in extreme cases, the oral care agent can be inoperative in absence of the activating agent; more typically the oral agent exhibits greater or faster efficacy, or improved substantivity, with than without the activating agent, as determinable by in vitro or in vivo testing.

An “activating effective amount” of an activating agent is an amount sufficient to promote effectiveness of an oral care agent as defined above.

A “basifying agent” herein is an agent that raises pH in the immediate environment of a surface to which it is applied. The pH can be, but is not necessarily, raised to a value of about 7 or higher from an initially acidic pH level.

An “orally acceptable” compound, composition or vehicle is one that is not harmful to a mammal in amounts disclosed herein when retained in the mouth, without swallowing, for a period sufficient to permit application to an oral surface as required herein. In general, such a compound, composition or vehicle is not harmful even if unintentionally swallowed.

The method of the invention can be seen to comprise at least two steps, wherein a first step involves preconditioning an oral surface, for example a dental surface, by wiping the surface with an absorbent fabric having impregnated therein or coated thereon an orally acceptable preconditioning agent, for example an activating agent.

The nature and composition of the fabric is not critical. The fabric can be woven (e.g., of tight or open weave) or nonwoven and is suitably composed of fibers, for example, cellulose fibers derived from any suitable vegetable source such as cotton or woodpulp. The fibers themselves and pores or interstices between the fibers can provide,loci for retention of the activating agent and, if desired, other substances. Upon wiping a surface with the fabric, a portion of the agent impregnated therein or coated thereon is transferred to the surface being wiped.

Conveniently, the fabric can be in a form of small swab or swatch adapted for one-time use. Such a swab or swatch can be an essentially laminar article such as a paper towelette or a more isodiametric article such as a cotton ball. Optionally one or more of such articles can be mounted on a more or less nonabsorbent structure such as a stick, for example resembling a Q-tips® cotton swab.

Optionally a towelette can have a more or less nonabsorbent backing sheet for ease or comfort in handling, thus in this case the towelette has an absorbent face and a nonabsorbent (backing or handling) face. In such a case, it is desirable that the absorbent face of the towelette be readily distinguishable by sight (e.g., color) and/or touch (e.g., texture) from the nonabsorbent face, so that in use the absorbent face is directed toward the oral or dental surface.

In one embodiment the fabric is provided in a form of a moist towelette or wet wipe, wherein the preconditioning agent is dissolved or dispersed in a liquid medium. In another embodiment the fabric is provided in a form of a substantially dry towelette or dry wipe, from which most or all of the water or other liquid medium previously used to coat or impregnate the fabric has been removed, for example by evaporation. According to this latter embodiment, transfer of the preconditioning agent from the fabric to the oral surface relies upon moisture present in the mouth:

It is noted that use of a dry towelette or wipe not only can deliver the preconditioning agent but also can result usefully in removal of excess saliva from the oral surface prior to application of the oral care agent.

Whether moist or dry, the towelette or wipe can be supplied in a sealed multiple or individual package, for example a plastic or foil wrapper, which is removed before use.

In an embodiment of the present method, the preconditioning step is performed with a wiping means, wherein the wiping means includes a coated or impregnated fabric in any form disclosed herein and equivalents thereof. Similarly, in an embodiment of the kit of the present invention, the coated or impregnated fabric component of the kit is a wiping means as defined immediately above.

The fabric can have an activating agent and other optional substances impregnated therein, i.e., occupying pores and interstices in the fabric. Alternatively or in addition, a coating or layer, for example of a semi-solid formulation comprising an activating agent and other optional substances, can be present on one or more surfaces of the fabric. A recitation herein of a fabric, for example a towelette or wipe, “containing” an activating agent or other substance will be understood to embrace a fabric impregnated and/or coated with such agent or substance.

Any suitable process known in the art can be used to provide a coating on the fabric. For example a coating can be applied by spraying, brushing or rolling, as in applying paint. Typically a coating composition having the preconditioning agent and other optional ingredients is applied to the surface of the fabric in diluted form in a liquid vehicle, for example water, ethanol or a mixture thereof. The fabric is then partially or completely dried to provide a coated fabric useful in the method of the invention.

Likewise, any suitable process known in the art can be used to impregnate the fabric with the preconditioning agent and other optional substances. For example, the fabric can be bathed in a liquid (e.g., aqueous and/or ethanolic) solution or dispersion of the desired substances, or a metered volume of such a solution or dispersion can be dispensed onto the fabric, for example by spraying or pipetting. Such a solution or dispersion can alternatively be transferred to the fabric by blotting, sponging or rolling. In any of the above processes, the fabric can then be packaged without drying, or partially or completely dried before packaging.

In yet another process, the fabric can be passed through a dry bath containing the preconditioning agent in solid form, followed by rolling to mechanically impregnate the agent into the fabric.

In one embodiment the subsequently applied oral care agent is pH-dependently activatable, and the preconditioning or activating agent is a pH modifying agent. Where the oral care agent is one that performs best when placed in an alkaline environment, for example a peroxy compound used for whitening a dental surface, a suitable activating agent is a basifying agent, for example an alkali metal carbonate or bicarbonate such as sodium bicarbonate, or an alkali or alkaline earth metal hydroxide such as sodium or calcium hydroxide. Where the oral care agent subsequently applied is one that performs best when placed in an acid environment, for example a fluoride salt used as an anti-caries treatment or a bicarbonate salt used to provide a clean mouth feel through effervescence, a suitable activating agent is an acidifying agent, for example an organic or inorganic acid or acid salt such as hydrochloric, phosphoric or citric acids. Thus in either case a suitable activating agent is one that provides a pH environment at the oral surface favorable to activation of the oral care agent.

Optionally a suitable buffering agent is present along with the pH modifying agent, and/or in some cases the pH modifying agent can itself have buffering activity.

As illustration, wiping a dental surface or simulated dental surface with a moist towelette impregnated with sodium bicarbonate has been found to be surprisingly effective in providing a surface environment of high pH. Effectiveness of a basifying agent-impregnated towelette in this regard can be determined by a test conducted substantially as follows.

A solution of a basifying agent is prepared at a concentration of about 1% to about 40% by weight in an aqueous vehicle, to provide a test solution. The aqueous vehicle can consist essentially of water or can contain one or more additional solvents such as ethanol and, optionally, other ingredients such as a humectant. Then, to a dry towelette is applied a measured volume of the test solution, not to exceed the absorbent capacity of the towelette. Alternatively, the towelette can be soaked to its full absorbent capacity with the test solution. Either way, the towelette becomes impregnated with the basifying agent.

The towelette can be used with or without partial or complete drying following impregnation with the basifying agent.

A suitable substrate simulating a human dental surface, for example synthetic hydroxyapatite disks or bovine teeth, is washed with water and then wiped with the impregnated towelette. A suitable indicator, e.g., phenolphthalein, can be used to determine whether a threshold pH, e.g., pH about 9 in the case of phenolphthalein, has been reached at the treated surface.

Similar testing protocols using appropriate indicators will readily be devised by one of skill in the art, based on the present disclosure, for evaluation of an acidifying agent-impregnated towelette.

The preconditioning or activating agent can be other than a pH modifying agent. For example, in one embodiment where the oral care agent is a peroxy compound, the preconditioning or activating agent comprises a compound or complex comprising iron, copper or manganese, for example manganous chloride, manganous citrate, manganese gluconate, ferrous sulfate, copper sulfate, mixtures thereof and the like.

In another embodiment the preconditioning or activating agent is a polymer that enhances substantivity of the subsequently applied oral care agent. For example, where the oral care agent is an antibacterial agent, any one or more of certain polymers present in the towelette or other coated or impregnated fabric can lead to greater longevity of antibacterial activity in the immediate environment of the treated surface. Polymers providing enhancement of antibacterial activity and/or substantivity are referred to herein as “antibacterial enhancing agents” (AEAs).

Polymers useful as AEAs include for example oligomers, homopolymers, copolymers and the like. The AEA can be natural or synthetic, and can be water insoluble or water soluble or swellable. In one embodiment the AEA is hydratable or hydrogel forming in saliva. Typically an AEA has a weight average molecular weight of about 100 to about 1,000,000, for example about 1,000 to about 1,000,000, or about 2,000 to about 500,000, or about 2,500 to about 250,000.

AEAs have a chemical structure that contains at least one delivery-enhancing group, and at least one organic retention-enhancing group. A “delivery-enhancing group” is one that attaches or substantively, adhesively, cohesively or otherwise bonds the AEA to an oral (e.g. dental) surface. An “organic retention-enhancing group”, generally hydrophobic, attaches or otherwise bonds a subsequently applied antibacterial agent to the AEA, thereby promoting retention of the antibacterial agent on the oral surface.

Illustratively, the AEA is an anionic polymer, for example a polycarboxylate comprising a chain or backbone containing repeating units each having at least one carbon atom and at least one directly or indirectly pendent monovalent delivery-enhancing group and at least one directly or indirectly pendent monovalent retention-enhancing group geminally, vicinally or otherwise bonded to atoms in the chain or backbone. Such polycarboxylates can be employed in free acid form or as partially or fully neutralized alkali metal (e.g., potassium, sodium) or ammonium salts. Further examples are 1:4 to 4:1 copolymers of maleic anhydride or acid with another polymerizable ethylenically unsaturated monomer such as methyl vinyl ether, having a molecular weight of about 30,000 to about 1,000,000, for example about 30,000 to about 500,000. Polyvinyl methyl ether/maleic anhydride (PVME MA) copolymers are available for example under the Gantrez™ brand from ISP, Wayne, N.J., e.g., Gantrez™ S-97.

The towelette, swab or other fabric used in the first step of the present method can have coated thereon or impregnated therein additional ingredients besides the preconditioning agent. For example, a moist towelette can have a humectant such as glycerin or sorbitol along with the preconditioning agent, to help keep the towelette moist.

A fabric useful in the present method, for example a moist or dry towelette, can optionally have at least one flavorant therein or thereon. Any orally acceptable natural or synthetic flavorant can be used, such as oils, aldehydes, esters, alcohols and the like, and mixtures, including multi-component mixtures, thereof. Flavorants include without limitation vanillin, sage, marjoram, parsley oil, spearmint oil, cinnamon oil, oil of wintergreen (methyl salicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamide, 3-1-menthoxypropane-1,2-diol, N,2,3-trimethyl-2-isopropyl-butanamide, cinnamaldehyde glycerol acetal (CGA), menthone glycerol acetal (MGA), capsicum, benzyl nicotinate and the like.

Incorporation of a suitable flavorant in the towelette or other fabric can be highly beneficial. It is likely that some users will find the act of wiping an oral surface, for example wiping the teeth, with a moist or dry towelette containing a bicarbonate salt or other activating agent uncomfortable or unpleasant. Presence in the towelette of a flavorant can greatly enhance sensory acceptability of the wiping step to such users, and thereby improve user compliance.

The duration of wiping and the intensity of lateral motion (rubbing), if any, during wiping that provides effective preconditioning of the oral surface can be determined by one of skill in the art without undue experimentation. It can be expected that a moist towelette will require a shorter duration of contact with the surface than a dry towelette, but in either case wiping for about 3 to about 300 seconds, for example about 5 to about 120 seconds or about 10 to about 60 seconds, will often suffice.

In a further embodiment, an article useful in preconditioning a dental surface for whitening treatment is provided. The article comprises a moist towelette carrying in aqueous solution an orally acceptable basifying agent, for example sodium bicarbonate, in an amount effective to provide a pH of at least about 7 at the dental surface upon wiping the surface with the towelette. Such a towelette is sufficiently moist that the basifying agent is maintained to a substantial degree in dissolved form and is therefore substantially non-abrasive when applied to the dental surface.

The towelette of this embodiment is typically packaged in a single-use substantially water-impermeable wrapper, for example a plastic or foil sachet. As described above, other ingredients can be present along with the basifying agent, including for example one or more humectant(s) and flavorant(s).

A second step of the method of the present invention involves applying a composition comprising an oral care agent, for example a peroxy compound, a fluoride ion source or a bicarbonate salt, to the oral surface after preconditioning as described above.

Such a composition can be, for example, a mouthwash or rinse, an oral spray, a dentifrice, an oral strip, a liquid whitener or a chewing gum. Rinses include liquids adapted for irrigation by means of devices such as high-pressure water jets. Dentifrices include without limitation toothpastes, gels and powders. A “liquid whitener” herein encompasses semi-liquid compositions such as gels as well as flowable liquids, so long as the composition is capable of application to a dental surface by painting with a brush or other suitable device. “Painting” in the present context means application of a thin layer of the composition to the dental surface, as is directed, for example, on the packaging of Colgate® Simply White® Night clear whitening gel sold by Colgate-Palmolive Co., New York, N.Y.

Classification herein of an ingredient as an active or a carrier ingredient is made for clarity and convenience, and no inference should be drawn that a particular ingredient necessarily functions in the composition in accordance with its classification herein. Furthermore, a particular ingredient can serve a plurality of functions, thus disclosure of an ingredient herein as exemplifying one functional class does not exclude the possibility that it can also exemplify another functional class.

Among useful oral care actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, a composition useful in the method and kit of the invention can contain one or more actives such as whitening agents, abrasives, anticalculus (tartar control) agents, fluoride ion sources, stannous ion sources, zinc ion sources, antimicrobial agents, antioxidants, sialagogues, breath freshening agents, antiplaque agents, anti-inflammatory agents, desensitizing agents, periodontal agents, analgesics and nutrients.

Actives useful herein are normally present in the composition in amounts selected to be safe and effective. A “safe and effective” amount in the present context is an amount sufficient to provide a desired benefit, for example a therapeutic, prophylactic or cosmetic effect, when the composition is used repeatedly as described herein, without undue side effects such as toxicity, irritation or allergic reaction, commensurate with a reasonable benefit/risk ratio. Such a safe and effective amount will usually, but not necessarily, fall within ranges approved by appropriate regulatory agencies. A safe and effective amount in a specific case depends on many factors, including the particular benefit desired or condition being treated or sought to be prevented, the particular subject using, or being administered, the composition, the frequency and duration of use, etc.

Among useful carriers are diluents, bicarbonate salts, pH modifying agents, surfactants, foam modulators, stabilizing agents for particular oral care actives including peroxide stabilizers, thickening agents, viscosity modifiers, mouth feel modifying agents, humectants, sweeteners, flavorants and colorants. One carrier material, or more than one carrier material of the same or different classes, can optionally be present.

In one embodiment the composition comprises as a whitening agent at least one peroxy compound, optionally together with one or more additional whitening agents such as chlorine dioxide, chlorites and hypochlorites (e.g., chlorites and hypochlorites of alkali and alkaline earth metals such as lithium, potassium, sodium, magnesium, calcium and barium). Suitable peroxy compounds include hydrogen peroxide, peroxides of alkali and alkaline earth metals, organic peroxy compounds and peroxy acids and salts thereof. Any orally acceptable compound that delivers a perhydroxy (OOH⁻) ion is useful. A peroxy compound can optionally be present in a form of a polymer-peroxide complex, for example a polyvinylpyrrolidone-hydrogen peroxide complex.

Peroxides of alkali and alkaline earth metals include lithium peroxide, potassium peroxide, sodium peroxide, magnesium peroxide, calcium peroxide and barium peroxide.

Organic peroxy compounds include, for example, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like.

Peroxy acids and their salts include organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium. Another useful peroxy compound is sodium pyrophosphate peroxyhydrate.

The whitening agent is present in the composition of the present embodiment in a total amount effective to result in whitening of a dental surface when applied in accordance with the disclosure herein. Peroxy compounds can illustratively be present in a total hydrogen peroxide equivalent amount of about 0.1% to about 10%, for example about 1% to about 5%, by weight of the composition.

As indicated above, where the oral care agent present in the composition is a peroxy compound, the fabric used for preconditioning the oral surface can contain an activating agent for the peroxy compound, such as a basifying agent, or an iron, copper or manganese compound or complex.

In a further embodiment the composition comprises a source of fluoride ions, such as a fluoride, monofluorophosphate or fluorosilicate salt. Any such salt that is orally acceptable can be used, including without limitation alkali metal (e.g., potassium, sodium), ammonium, stannous and indium salts and the like. Water-soluble fluoride-releasing salts are typically used. One or more fluoride-releasing salts are present in the composition of this embodiment in an amount providing a total of about 100 to about 20,000 ppm, about 200 to about 5,000 ppm, or about 500 to about 2,500 ppm, fluoride ions. Where sodium fluoride is the sole fluoride-releasing salt present, illustratively an amount of about 0.01% to about 5%, about 0.05% to about 1% or about 0.1% to about 0.5%, sodium fluoride by weight can be present in the composition.

As indicated above, where the oral care agent present in the composition is a source of fluoride ions, the fabric used for preconditioning the oral surface can contain an activating agent for the fluoride source, such as an acidifying agent.

In a further embodiment the composition comprises at least one bicarbonate salt, useful for example to impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate and the like. One or more bicarbonate salts are optionally present in a total amount of 0.1% to about 50%, for example about 1% to about 20% by weight of the composition.

As indicated above, where the oral care agent present in the composition is a bicarbonate salt, the fabric used for preconditioning the oral surface can contain an activating agent for the peroxy compound, such as an acidifying agent, to promote effervescence.

The composition can optionally comprise at least one abrasive, useful for example as a cleaning and/or polishing agent. Any orally acceptable abrasive can be used, but type, fineness (particle size) and amount of abrasive should be selected so that tooth enamel is not excessively abraded in normal use of the composition. Suitable abrasives include without limitation silica, for example in the form of silica gel, hydrated silica, pyrogenic silica or precipitated silica, alumina, for example in the form of hydrated alumina or calcined alumina, aluminum silicate, bentonite, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. One or more abrasives are optionally present in the composition in an abrasive effective total amount, typically about 5% to about 70%, for example about 10% to about 50% or about 15% to about 30% by weight. Average particle size of an abrasive, if present, is generally about 0.1 to about 30 μm, for example about 1 to about 20 μm or about 5 to about 15 μm.

Among the above abrasives, siliceous and/or aluminous abrasives including silica, hydrated silica, pyrogenic silica, silica gels and precipitates, alumina, hydrated alumina, calcined alumina, aluminum silicate and bentonite, when used in abrasive effective amounts, are typically incompatible with peroxy compounds, in large measure because of transition metal impurities that can be present in mineral products such as these. Such incompatible abrasives should therefore be avoided where a peroxy compound is included in the composition. Abrasives such as insoluble phosphates that are not incompatible with peroxy compounds can, if desired, be included in a peroxy compound-containing composition.

In a particular embodiment one or more siliceous and/or aluminous abrasives, for example hydrated silica, are present in a total amount of about 15% to about 30% by weight of the composition.

The composition can optionally include a first abrasive selected primarily for high cleaning efficacy and a second abrasive selected primarily for polishing efficacy and/or enhanced mouth feel. Such first and second abrasives are herein termed “high-cleaning” and “prophy” abrasives respectively. For example, a high-cleaning silica and a prophy silica can be included, each illustratively in a total amount of about 5% to about 15% by weight of the composition.

The composition can optionally -comprise at least one antimicrobial (e.g., antibacterial) agent. Any orally acceptable antimicrobial agent can be used, including without limitation triclosan (5-chloro-2-(2,4-dichlorophenoxy)phenol), 2,2′-dihydroxy-5,5′-dibromodiphenyl ether, 8-hydroxyquinoline and salts thereof, copper (II) compounds such as copper (II) chloride, fluoride, sulfate and hydroxide, zinc ion sources such as zinc citrate, zinc sulfate, zinc glycinate and sodium zinc citrate, phthalic acid and salts thereof such as magnesium monopotassium phthalate, hexetidine, octenidine, sanguinarine, benzalkonium chloride, salicylanilide, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, octenidine, iodine, sulfonamides, bisbiguanides such as alexidine, chlorhexidine and chlorhexidine digluconate, phenolics, piperidino derivatives such as delmopinol and octapinol, magnolia extract, grapeseed extract, phenol, thymol, eugenol, menthol, geraniol, carvacrol, citral, eucalyptol, catechol, 4-allylcatechol, hexyl resorcinol, halogenated bisphenolics such as 2,2′-methylene bis(4-chloro-6-bromophenol), methyl salicylate, antibiotics such as augmentin, amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al., incorporated herein by reference. One or more antimicrobial agents are optionally present in an antimicrobial effective total amount, typically about 0.05% to about 3%, for example about 0.1% to about 1% by weight, of the composition.

Among antimicrobial agents, nonionic agents such as halogenated diphenylethers (e.g., triclosan and 2,2′-dihydroxy-5,5′-dibromodiphenyl ether) and phenolic compounds are typically incompatible with peroxy compounds and should therefore be avoided where a peroxy compound is included in the composition.

As indicated above, where the oral care agent present in the composition is a noncationic antibacterial agent, the fabric used for preconditioning the oral surface can contain an AEA, such as a PVME/MA copolymer, to promote substantivity of the antibacterial agent.

The composition can optionally comprise at least one anticalculus agent. Any orally acceptable anticalculus agent can be used, including without limitation phosphates and polyphosphates (for example pyrophosphates), polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium. Other useful anticalculus agents include PVME/MA copolymers, such as those available under the Gantrez™ brand from ISP, Wayne, N.J. One or more anticalculus agents are optionally present in the composition in an anticalculus effective total amount, typically about 0.01% to about 50%, for example about 0.05% to about 25% or about 0.1% to about 15% by weight.

In a particular embodiment one or more PVME/MA copolymers are present in a total amount of about 0.3% to about 3% by weight of the composition, optionally together with one or more polyphosphate salts, e.g., tetrasodium pyrophosphate, tetrapotassium pyrophosphate, sodium tripolyphosphate and/or potassium tripolyphosphate, in a total amount of about 1% to about 15% by weight.

The composition can optionally comprise at least one stannous ion source useful, for example, in helping reduce gingivitis, plaque, calculus, caries or sensitivity. Any orally acceptable stannous ion source can be used, including without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5%, by weight of the composition.

The composition can optionally comprise at least one zinc ion source useful, for example, as an antimicrobial, anticalculus or breath-freshening agent. Any orally acceptable zinc ion source can be used, including without limitation zinc citrate, zinc sulfate, zinc glycinate, sodium zinc citrate and the like. One or more zinc ion sources are optionally and illustratively present in a total amount of about 0.05% to about 3%, for example about 0.1% to about 1%, by weight of the composition.

The composition can optionally comprise at least one antioxidant. Any orally acceptable antioxidant can be used, including without limitation butylated hydroxyanisole (BHA); butylated hydroxytoluene (BHT), vitamin A, carotenoids, vitamin E, flavonoids, polyphenols, ascorbic acid, herbal antioxidants, chlorophyll, melatonin and the like. One or more antioxidants are optionally present in an antioxidant effective total amount. In a particular embodiment at least one of BHA and BHT is present in the composition in a total amount of about 0.01% to about 0.1% by weight.

The composition can optionally comprise a sialagogue (saliva stimulating agent), useful for example in amelioration of dry mouth. Any orally acceptable sialagogue can be used, including without limitation food acids such as citric, lactic, malic, succinic, ascorbic, adipic, fumaric and tartaric acids. One or more sialagogues are optionally present in the composition in a saliva stimulating effective total amount.

The composition can optionally comprise a breath freshening agent. Any orally acceptable breath freshening agent can be used, including without limitation zinc salts such as zinc gluconate, zinc citrate and zinc chlorite, α-ionone and the like. One or more breath freshening agents are optionally present in the composition in a breath freshening effective total amount.

The composition can optionally comprise an antiplaque, including plaque disrupting, agent. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and chelating agents such as citric and tartaric acids and alkali metal salts thereof. One or more antiplaque agents are optionally present in the composition in an antiplaque effective total amount.

The composition can optionally comprise at least one anti-inflammatory agent. Any orally acceptable anti-inflammatory agent can be used, including without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in the composition in an anti-inflammatory effective amount.

The composition can optionally comprise at least one desensitizing agent. Potassium salts such as potassium citrate, potassium tartrate, potassium chloride, potassium sulfate and potassium nitrate are illustratively useful in this regard, as is sodium nitrate. Alternatively or in addition a local or systemic analgesic such as aspirin, codeine, acetaminophen, sodium salicylate or triethanolamine salicylate can be used. One or more desensitizing agents and/or analgesics are optionally present in the composition in a desensitizing and/or analgesic effective amount.

The composition can optionally comprise at least one nutrient. Suitable nutrients include vitamins, minerals and amino acids.

The composition can optionally comprise at least one thickening agent, useful for example to impart a desired consistency and/or mouth feel to the composition. Thickening agents include organic, clay-based and colloidal silica thickening agents. Any orally acceptable organic thickening agent can be used, including without limitation carbomers, also known as carboxyvinyl polymers, for example those sold under the Carbopol™ brand including Carbopols 934, 956, 974 and 980, polyvinylpyrrolidone, carrageenans, also known as Irish moss and more particularly i-carrageenan (iota-carrageenan), cellulosic polymers such as hydroxyethylcellulose, hydroxypropylmethylcellulose, carboxymethylcellulose (CMC) and carboxymethyl-hydroxyethylcellulose and salts thereof, e.g., CMC sodium, starches, and natural gums such as karaya, xanthan, gum arabic and tragacanth. Any orally acceptable clay-based thickening agent can be used, including such agents comprising natural, modified and/or synthetic clays. Illustratively, thickening agents comprising at least one clay of the smectite class, including beidellite, bentonite, hectorite, montmorillonite, saponite and stevensite, and synthetic counterparts such as colloidal magnesium aluminum silicate and Laponite™ are useful. Hydrophobically modified clays such as hydrophobically modified bentonite are also useful. One or more thickening agents are optionally present in a total amount of about 0.01% to about 15%, for example about 0.1% to about 10% or about 0.2% to about 5% by weight of the composition.

The composition can optionally comprise at least one viscosity modifier, useful for example to inhibit settling or separation of ingredients or to promote redispersibility upon agitation of a liquid composition. Any orally acceptable viscosity modifier can be used, including without limitation mineral oil, petrolatum, clays and organomodified clays, silica and the like. One or more viscosity modifiers are optionally present in a total amount of about 0.01% to about 10%, for example about 0.1% to about 5% by weight of the composition.

The composition can optionally comprise at least one pH modifying agent. Such agents include acidifying agents to lower pH, basifying agents to raise pH and buffering agents to control pH within a desired range. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain the composition in a desired pH range.

The composition can optionally comprise at least one surfactant, useful for example to compatibilize other ingredients and thereby provide enhanced stability, to help in cleaning the dental surface through detergency, and to provide foam upon agitation, e.g., during brushing. Any orally acceptable surfactant, including cationic, anionic, nonionic and amphoteric types, can be used.

Suitable cationic surfactants include without limitation quaternary ammonium compounds with a C₈₋₂₀ aliphatic chain such as lauryl trimethylammonium chloride, cetyl pyridinium chloride, cetyl pyridinium fluoride, cetyl trimethylammonium bromide, diisobutylphenoxyethyl-dimethylbenzylammonium chloride, cocoalkyltrimethylammonium nitrite and the like. Cationic compounds that can stain teeth, for example chlorhexidine, can be considered for use herein, bearing this disadvantage in mind.

Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium lauryl sulfoacetate, sodium laureth carboxylate, sodium dodecyl benzenesulfonate and sodium and potassium salts of lauroyl sarcosinate, myristoyl sarcosinate, palmitoyl sarcosinate, stearoyl sarcosinate and oleoyl sarcosinate.

Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like.

Suitable amphoteric surfactants include without limitation derivatives of C₈₋₂₀ aliphatic secondary and tertiary amines having an anionic group such as carboxylate, sulfate, sulfonate, phosphate or phosphonate. Examples include cocoamidopropyl betaine and lauramidopropyl betaine.

One or more surfactants are optionally present in a total amount of about 0.01% to about 10%, for example about 0.05% to about 5% or about 0.1% to about 2% by weight of the composition.

The composition can optionally comprise at least one foam modulator, useful for example to increase amount, thickness or stability of foam generated by the composition upon agitation, e.g., brushing. Any orally acceptable foam modulator can be used, including without limitation polyethylene glycols (PEGs), also known as polyoxyethylenes. High molecular weight PEGs are suitable, including those having an average molecular weight of about 200,000 to about 7,000,000, for example about 500,000 to about 5,000,000 or about 1,000,000 to about 2,500,000. One or more PEGs are optionally present in a total amount of about 0.1% to about 10%, for example about 0.2% to about 5% or about 0.25% to about 2% by weight of the composition.

The composition can optionally comprise at least one humectant, useful for example to prevent hardening of the composition upon exposure to air, and/or to enhance mouth feel. Any orally acceptable humectant can be used, including without limitation polyhydric alcohols such as propylene glycol, butylene glycol, glycerin, sorbitol, xylitol or low molecular weight PEGs. Most humectants also function as sweeteners. One or more humectants are optionally present in a total amount of about 1% to about 80%, for example about 5% to about 65% or about 10% to about 50% by weight of the composition.

In a particular embodiment, the composition is a gel that comprises glycerin in an amount of about 10% to about 60% by weight, optionally together with a low molecular weight PEG such as PEG 600 in an amount of about 2% to about 20% by weight of the composition.

In another particular embodiment, the composition is a paste that comprises sorbitol in an amount of about 10% to about 50%, optionally together with glycerin in an amount of about 5% to about 25% by weight of the composition.

The composition can optionally comprise at least one sweetener, useful for example to enhance taste of the composition. Any orally acceptable natural or artificial, nutritive or non-nutritive sweetener can be used, including without limitation dextrose, polydextrose, sucrose, maltose, dextrin, dried invert sugar, lactose, mannose, xylose, ribose, fructose, galactose, corn syrup (including high fructose corn syrup and corn syrup solids), partially hydrolyzed starch, hydrogenated starch hydrolysate, sorbitol, mannitol, xylitol, maltitol, isomalt, sucralose, aspartame, acesulfame, neotame, D-tryptophan, saccharin and salts thereof (e.g., sodium saccharin), thaumatin, dihydrochalcones, dipeptide-based intense sweeteners, cyclamates (e.g., sodium cyclamate) and the like. One or more sweeteners are optionally present in a total amount depending strongly on the particular sweetener(s) selected, but typically about 0.005% to about 5% by weight of the composition.

In a particular embodiment, the composition comprises sodium saccharin in an amount of about 0.1% to about 1% by weight.

The composition can optionally comprise at least one flavorant, useful for example to enhance taste of the composition. Any orally acceptable natural or synthetic flavorant can be used, including without limitation those listed above as optional ingredients of the coated or impregnated fabric. One or more flavorants are optionally present in a total amount of about 0.01% to about 5%, for example about 0.1% to about 2.5% by weight of the composition.

The composition can optionally comprise at least one colorant. Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of about 0.001% to about 20%, for example about 0.01% to about 10% or about 0.1% to about 5% by weight of the composition.

In a particular embodiment, the composition can comprise two or more components having contrasting colors to provide a striped effect upon extrusion from a tube or other dispenser. For example, the composition can comprise a gel component containing a blue colorant and a paste component containing titanium dioxide to appear white.

A gel composition can be prepared by mixing the ingredients in any suitable mixing device. A paste composition can be prepared by the following general procedure. Water and thickening agent(s), typically together with humectant(s) and sweetening agent(s), are mixed in a suitable mixing device until a homogeneous gel phase is obtained. Into the gel phase other ingredients, such as pigment(s) and fluoride ion source(s), can be added with further mixing until homogeneous. Thereafter, abrasive(s) and/or other desired ingredients such as anticalculus agent(s), antibacterial agent(s), flavorant(s) and surfactant(s) are added and the resulting mixture is mixed at high speed, optionally under vacuum of about 20 to about 100 mm Hg, to provide a homogeneous extrudable paste.

The oral surface to be treated by the method of the invention can be in a human or nonhuman subject, for example a nonhuman mammalian subject such as a companion animal, for example a dog or cat. In one embodiment the oral surface is a surface of one or more natural teeth, but the method is also applicable to a surface of artificial dentition, for example a crown, a cap, a filling, a bridge, a denture or a dental implant.

Practice of the method can consist of a single two-step application as described herein, or can comprise repeated such applications. In one embodiment the present method is repeated at regular intervals, for example twice or once daily, twice or once weekly, twice or once monthly, in a program or regimen conducted at home and/or in a professional or clinical setting.

Increase in whiteness of a dental surface can be observed visually, for example with the aid of color comparison charts, gauges or shade guides, e.g., as described by Browning (2003), Journal of Esthetic Restorative Dentistry 15 Supp. 1, S13-S20, incorporated herein by reference.

Alternatively, increase in whiteness can be measured by colorimetry, using any suitable instrument such as a Minolta Chromameter, e.g., model CR-321 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to measurement of tooth whiteness. Increase in whiteness can be computed from differences in L*, a* and b* values before and after treatment, or between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}
A higher value of ΔE* indicates greater increase in whiteness. In various embodiments, the method of the present invention can effect a ΔE* of at least about 1, or at least about 3, or at least about 5.

Evaluation of effectiveness of whitening treatments of the invention can be made, for example, in clinical studies using human volunteers, or in vivo in animals, conducted according to appropriate protocols.

Suitable in vitro protocols are also available for evaluation of whitening treatments, including those described in Examples herein and in published literature. See for example Stookey et al. (1982), Journal of Dental Research 61(11), 1236-1239, and Rice et al. (2001), Journal of Clinical Dentistry 12(2), 34-37, both incorporated herein by reference.

A kit of the invention comprises a composition comprising an oral care agent, for example a whitening composition comprising an orally acceptable peroxy compound. Such a composition, as described above, can illustratively be a mouthwash or rinse, an oral spray, a gel or paste dentifrice, an oral strip, a liquid whitener or a chewing gum. The composition is typically supplied in suitable packaging, for example a dispensing container such as a tube or pump where the composition is a dentifrice.

The kit further comprises an absorbent fabric, for example in a form of a moist or dry towelette, having impregnated therein or coated thereon an orally acceptable preconditioning agent. The preconditioning agent can be an activating agent for the oral care agent, for example a basifying agent such as an alkali metal carbonate or bicarbonate salt, e.g., sodium bicarbonate, where the oral care agent is a peroxy compound.

The fabric and oral care agent composition components of the kit can be packaged separately or together and can be sold individually or as a single product. Typically instructions for use of the kit according to the method of the present invention are provided.

The kit is useful for practice of the invention in a professional setting (e.g., a dentist's or dental hygienist's office or clinic) or by the user at home or while traveling. Although adapted particularly for human use, the kit can be useful for administering oral care to nonhuman animals, for example domestic pets such as dogs.

The invention can further be understood by reference to the following nonlimiting examples.

### EXAMPLES

### Example 1

Solutions A-I were prepared having the composition shown in Table 1. Each of these solutions could be dispersed uniformly on a towelette.

TABLE 1
Composition of solutions A-I

| | Weight % | | | | | | | | |
|---|---|---|---|---|---|---|---|---|---|
| Ingredient | A | B | C | D | E | F | G | H | I |
| ethanol | 5 | 5 | 5 | 5 | 5 | 5 | 5 | 5 | 5 |
| glycerin | 50 | 50 | 50 | 50 | 50 | 50 | 50 | 50 | 50 |
| sodium carbonate | 5 | | | | | | | | |
| sodium bicarbonate | | 5 | | | | | | | 10 |
| sodium hydroxide | | | 5 | | | | | | |
| calcium hydroxide | | | | 5 | | | | | |
| phosphoric acid | | | | | 5 | | | | |
| hydrochloric acid | | | | | | 5 | | | |
| citric acid | | | | | | | 5 | | |
| Gantrez ® | | | | | | | | 5 | |
| flavoring agent | 2 | 2 | 2 | 2 | 2 | 2 | 2 | 2 | 2 |
| water | q.s. | q.s. | q.s. | q.s. | q.s. | q.s. | q.s. | q.s. | q.s. |

A towelette moistened with solution A was tested for its effect on pH at the surface of synthetic hydroxyapatite (SHAP) disks. Initially, SHAP disks were washed with water to remove any fine particles and dust. The surface of the disks was then wiped with the moist towelette containing solution A. The pH of the surface was then tested using phenolphthalein indicator. The phenolphthalein turned pink, indicating a pH>9.

A similar test was conducted using bovine teeth as the substrate. Again wiping with a moist towelette containing solution A provided a surface pH>9 as indicated by phenolphthalein.

Similar results were obtained with towelettes moistened with solutions B, C and D.

### Example 2

Effectiveness of Colgate® Simply White® liquid whitener in whitening a simulated dental surface (coffee-stained eggshell or tea-stained Formica® laminate) was compared, with and without preconditioning of the surface by wiping with a moist towelette containing solution B of Example 1.

Eggs were initially cooked in boiling water for 20 minutes. After cooling, the eggs were soaked in coffee at room temperature for 30 minutes, and were then washed in water. Uniformly coffee-stained eggs were selected for testing, two eggs for each treatment. Three regions of each egg were demarcated. A first region was wiped with a towelette that had been soaked in solution B of Example 1. A second region was wiped with a water-soaked towelette (as a control). A third region was left untreated. Immediately thereafter, liquid whitener was applied to the first and second regions and left for 2.5 minutes followed by washing the egg in running tap water.

Degree of whitening at five locations in each of the first and second regions was measured by comparison with five locations in the third region. A Minolta CR-321 chromameter was used to determine color changes using the L*a*b* system as described above, where a higher value of ΔE* indicates greater increase in whiteness. Results are shown in Table 2.

TABLE 2
Increase in whiteness of eggshell

| Treatment | Average ΔE* |
|---|---|
| Wiped with water, then liquid whitener applied | 8.08 |
| Wiped with solution B, then liquid whitener applied | 19.20 |

The test was repeated on tea-stained laminate. Three applications of liquid whitener, each of 2 minutes duration, were made, each preceded by wiping with a towelette containing water or solution B. Chromameter results are shown in Table 3.

TABLE 3
Increase in whiteness of laminate

| Treatment | Average ΔE* |
|---|---|
| Wiped with water, then liquid whitener applied | 6.60 |
| Wiped with solution B, then liquid whitener applied | 13.28 |

On both surfaces, a substantial improvement in whitening was seen when the surfaces were preconditioned by wiping with a towelette containing solution B in accordance with the present invention. Improvement in whitening was also obtained with towelettes containing other basifying agents (solutions A, C and D). In these studies the order of effectiveness of basifying agents was as follows: sodium bicarbonate (B)>sodium carbonate (A)>sodium hydroxide (C)>calcium hydroxide (D).

1. A method for improving effectiveness of an oral care agent, the method comprising preconditioning an oral surface by wiping the surface with an absorbent fabric having impregnated therein or coated thereon an orally acceptable preconditioning agent, wherein said wiping transfers an effective amount of the preconditioning agent from the fabric to the oral surface, and thereafter applying a composition comprising the oral care agent to the oral surface

2. The method of claim 1, wherein the preconditioning agent is an activating agent for the oral care agent, and wherein said wiping transfers an activating effective amount of the activating agent from the fabric to the oral surface.

3. The method of claim 1 wherein the oral surface is a dental surface.

4. The method of claim 1 wherein the absorbent fabric is in a form of a towelette.

5. The method of claim 1 wherein the absorbent fabric further contains a flavorant.

6. The method of claim 1 wherein the oral care agent is pH-dependently activatable, and wherein the preconditioning agent is a pH modifying agent that provides a pH environment at the oral surface favorable to activation of the oral care agent.

7. The method of claim 6 wherein the oral care agent is a peroxy compound, and wherein the pH modifying agent is a basifying agent.

8. The method of claim 7 wherein the basifying agent is an alkali metal carbonate or bicarbonate salt.

9. The method of claim 7 wherein the basifying agent is sodium bicarbonate.

10. A method for whitening a dental surface, the method comprising wiping the surface with an absorbent fabric having impregnated therein or coated thereon an orally acceptable basifying agent, and thereafter applying a composition comprising an orally acceptable peroxy compound to the surface.

11. The method of claim 10 wherein the absorbent fabric is in a form of a towelette.

12. The method of claim 10 wherein the absorbent fabric further contains a flavorant.

13. The method of claim 10 wherein the basifying agent is an alkali metal carbonate or bicarbonate salt.

14. The method of claim 10 wherein the basifying agent is sodium bicarbonate.

15. The method of claim 10 wherein the peroxy compound is selected from the group consisting of hydrogen peroxide, peroxides of alkali and alkaline earth metals, organic peroxy compounds, peroxy acids and salts thereof, and polymer-peroxide complexes.

16. The method of claim 10 wherein the peroxy compound is hydrogen peroxide.

17. The method of claim 10 wherein the composition comprising the peroxy compound is a whitening strip.

18. The method of claim 10 wherein the composition comprising the peroxy compound is a liquid whitener.

19. The method of claim 10 wherein the composition comprising the peroxy compound is a semi-solid dentifrice.

20. A kit useful for oral care, the kit comprising (a) a composition that comprises an oral care agent, and (b) an absorbent fabric having impregnated therein or coated thereon an orally acceptable preconditioning agent.

21. The kit of claim 20, wherein the preconditioning agent is an activating agent for the oral care agent.

22. The kit of claim 20 wherein the absorbent fabric is in a form of a towelette.

23. The kit of claim 20 wherein the absorbent fabric further contains a flavorant.

24. The kit of claim 20 wherein the oral care agent is an orally acceptable peroxy compound and the preconditioning agent is a basifying agent.

25. The kit of claim 24 wherein the basifying agent is an alkali metal carbonate or bicarbonate salt.

26. The kit of claim 24 wherein the basifying agent is sodium bicarbonate.

27. The kit of claim 24 wherein the peroxy compound is selected from the group consisting of hydrogen peroxide, peroxides of alkali and alkaline earth metals, organic peroxy compounds, peroxy acids and salts thereof, and polymer-peroxide complexes.

28. The kit of claim 24 wherein the peroxy compound is hydrogen peroxide.

29. The kit of claim 24 wherein the composition comprising the peroxy compound is a whitening strip.

30. The kit of claim 24 wherein the composition comprising the peroxy compound is a liquid whitener.

31. The kit of claim 24 wherein the composition comprising the peroxy compound is a semi-solid dentifrice.

32. An article useful in preconditioning a dental surface for whitening treatment, the article comprising a moist towelette carrying in aqueous solution an orally acceptable basifying agent in an amount effective to provide a pH of at least about 7 at the dental surface upon wiping the surface with the towelette.

33. The article of claim 32 wherein the basifying agent is sodium bicarbonate.

34. The article of claim 32 wherein the towelette further contains a flavorant.
